(19) United States
(12) Patent Application Publication
(10) Pub. No.: US 2026/0061360 A1
(43) Pub. Date: Mar. 5, 2026

(54) CARBON CAPTURE SYSTEM FOR PRODUCTION OF SODA ASH, BAKING SODA, METHANOL, & FORMALDEHYDE

(71) Applicant: AGC Carbon Inc., Madison, CT (US)

(72) Inventor: Sumeet Gandhi, Madison, CT (US)

(73) Assignee: AGC Carbon Inc., Madison, CA (US)

(21) Appl. No.: 19/319,572

(22) Filed: Sep. 4, 2025

Related U.S. Application Data
(60) Provisional application No. 63/690,557, filed on Sep. 4, 2024.

(51) Int. Cl. B01D 53/14; B01D 53/18; B01D 53/48; B01D 53/54; B01D 53/62; B01D 53/75; B01D 53/78; B01D 53/96; C01C 1/02; C01C 1/16; C01D 7/12; C07C 29/151; C07C 31/04; C07C 45/00; C07C 47/04; C25B 1/01

(52) U.S. Cl. CPC B01D 53/1406; B01D 53/1418; B01D 53/1425; B01D 53/1475; B01D 53/1493; B01D 53/18; B01D 53/48; B01D 53/54; B01D 53/62; B01D 53/75; B01D 53/78; B01D 53/96; C01C 1/026; C01C 1/164; C01D 7/123; C07C 29/151; C07C 31/04; C07C 45/002; C07C 47/04; B01D 2251/304; B01D 2251/604; B01D 2252/102; B01D 2256/10; B01D 2256/12; B01D 2256/20; B01D 2257/30; B01D 2257/40; B01D 2257/504; B01D 2258/0283; C25B 1/01

(57) ABSTRACT

A carbon capture system is used to remove carbon dioxide from flue gas emissions. The system consists of a first scrubbing column, a carbonating tower, and a separation system. In the first scrubbing column, nitrates and sulfates are removed from the flue gas, producing a purified flue gas and a bottoms product containing the sulfates and nitrates. The purified flue gas is then transferred to a carbonating tower, where it is contacted with a solution (such as brine, ammonia, or a weak base) to remove carbon dioxide, producing a lean brine solution. The lean brine solution is then filtered to recover sodium bicarbonate or soda ash.

### CROSS-REFERENCE TO RELATED APPLICATIONS

This application claims the benefit of priority to U.S. provisional application No. 63/690,557, filed on Sep. 4, 2024, which is expressly incorporated by reference herein in its entirety.

### BACKGROUND

Many industrial operations produce byproducts that are found harmful to the environment. One byproduct from industrial applications that has been particularly important to remove from the environment is CO₂, a common off gas from industrial operations such as power plants, cement factories, and steel manufacturing facilities. These industries produce substantial amounts of CO₂ as a byproduct of combustion and chemical processes. One method for removing CO₂ is a carbon capture (CC) process where the carbon dioxide is separated and stored.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced. Details of one or more aspects of the subject matter described in this disclosure are set forth in the accompanying drawings and the description below. However, the accompanying drawings illustrate only some typical aspects of this disclosure and are therefore not to be considered limiting of its scope. Other features, aspects, and advantages will become apparent from the description, the drawings and the claims

FIG. 1 illustrates an example of a carbon capture system in accordance with some aspects of the subject matter of the present technology.

FIG. 2A illustrates the first part of an example of a carbon capture system that includes a modified Solvay process in accordance with some aspects of the subject matter of the present technology.

FIG. 2B illustrates the second part of an example of a carbon capture system that includes a modified Solvay process in accordance with some aspects of the subject matter of the present technology.

FIG. 3A illustrates the first part of an example of a carbon capture system that includes a modified Solvay process that introduces Aniline or Amine in accordance with some aspects of the subject matter of the present technology.

FIG. 3B illustrates the second part of an example of a carbon capture system that includes a modified Solvay process that introduces Aniline or Amine in accordance with some aspects of the subject matter of the present technology.

FIG. 4 illustrates a routine 400 in accordance with one embodiment.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

Carbon capture technologies typically capture CO₂ from fuel combustion and other industrial sources to be either pumped into the ground or converted into products that eventually release the CO₂ back to the environment. Different methods of carbon capture have been tried. For example, some carbon capture methods include absorbent-based systems, oxygen fuel combustion, and solid oxide fuel cells. However, these previous methods can actually lead to increasing the carbon footprint of the plants where they are installed because they rely on the use of carbon-based power systems, e.g., burning coal for electricity. These systems are not only carbon intensive, but also have capital expenditures and increase the operational costs of the plant because they have a large physical footprint that occupies a large portion of the plant or adjacent to the plant and require significant investment in utility upgrades to run the carbon capture systems. Further, these previous systems can produce significant wastewater and other side products, e.g., nitrous oxide, that have increased disposal and treatment costs. An alternate carbon capture system is needed that will reduce a system's carbon footprint, not require large storage additions to the process, and provide the energy source to power and operate the CC system.

The improved carbon capture (CC) system described below is a standalone, compact design that minimizes overall system footprint, thereby facilitating easier integration with existing infrastructure. This compatibility enhancement allows for minimal additional investment during installation, reducing upfront costs and increasing the system's economic viability. The CC system's ability to convert CO₂ into a valuable commodity is a significant advantage. Instead of the amine-based system, it is proposed that a modified Solvay process using a either a brine solution and/or a combination of brine solution with amine solution can be used for the production of soda ash. The addition of amine solution or alternatively a weak base to the ammoniated brine solution allows for the direct production of soda ash without the need to produce baking soda as an intermittent step. Furthermore, the CO₂ generated from the limestone calcination step is utilized to make methanol by hydrogenation of CO₂ in a gas diffusion membrane-based reactor cell utilizing a catalyst and UV light to reduce the temperature and pressure requirements for the hydrogenation reactor. The CO₂ fed to the CO₂ hydrogenation reactor can be either as gas or as a cryogenic liquid. The present disclosure also focuses further on hydrogenation of methanol to produce formaldehyde. Furthermore, while a brine solution is mentioned, the proposed system can also utilize a treated seawater solution for CO₂ absorption.

The present disclosure provides methods and systems for carbon capture and carbon utilization that integrate flue gas scrubbing, carbonate precipitation, ammonia recycle, and downstream chemical conversion into a unified process. In various embodiments, the invention provides both a method of capturing and converting carbon dioxide from flue gas and a carbon capture system configured to perform the method.

In one aspect, a method is provided that includes receiving a flue gas containing carbon dioxide, nitrogen, oxygen, nitrates, and sulfates at a carbon capture system; introducing the flue gas into a first scrubbing column to remove the nitrates and sulfates and generate a purified flue gas; transferring the purified flue gas to a carbonating tower; contacting the purified flue gas with an ammoniated brine or a brine solution including ammonia and, in some embodiments, a weak base; and precipitating sodium bicarbonate (NaHCO₃) while forming ammonium chloride (NH₄Cl) in solution. The NaHCO₃ is separated by filtration to provide a solid sodium bicarbonate product, while the NH₄Cl is recovered in the filtrate. The sodium bicarbonate can be further calcined to sodium carbonate (Na₂CO₃) with release of carbon dioxide, which can be recycled within the system or directed to further utilization processes. In some embodiments, the released carbon dioxide is introduced into a methanol reactor and hydrogenated to form methanol, optionally by electrochemical hydrogenation. In other embodiments, methanol produced by the methanol reactor is directed to an aldehyde reactor to yield formaldehyde.

In further aspects, the method includes processing the ammonium chloride recovered from the filtration step in an ammonia recovery column, where the NH₄Cl is reacted with calcium hydroxide [Ca(OH)₂] and steam to release ammonia (NH₃). The regenerated ammonia is returned to an ammonia absorber column along with brine and, in some embodiments, a weak base such as a primary amine, to generate an ammoniated brine that is continuously fed to the carbonating tower. The bottoms of the absorber column, containing ammonia and brine, are recycled back to the carbonation stage, thereby establishing a closed-loop ammonia cycle.

In another aspect, a carbon capture system is provided that includes a first scrubbing column for nitrate and sulfate removal, a carbonating tower for carbon dioxide removal, and a separation system for recovering sodium bicarbonate. In some embodiments, the system further includes a reactor for calcining sodium bicarbonate to sodium carbonate, a methanol reactor for hydrogenating carbon dioxide to methanol, and optionally an aldehyde reactor for converting methanol to formaldehyde. In still further embodiments, the separation system also recovers ammonium chloride, which is directed to an ammonia recovery column for regeneration of ammonia, and the regenerated ammonia is returned to an ammonia absorber column to produce an ammoniated brine for recycle to the carbonating tower.

By integrating these features, the disclosed methods and systems achieve carbon dioxide capture from flue gas in an efficient manner while producing commercially valuable co-products, including sodium bicarbonate, sodium carbonate, methanol, and formaldehyde, along with a recoverable ammonia loop and a calcium chloride byproduct stream. The current disclosure thereby enables both environmental benefits through reduction of greenhouse gas emissions and economic benefits through production of industrial chemicals in a unified process.

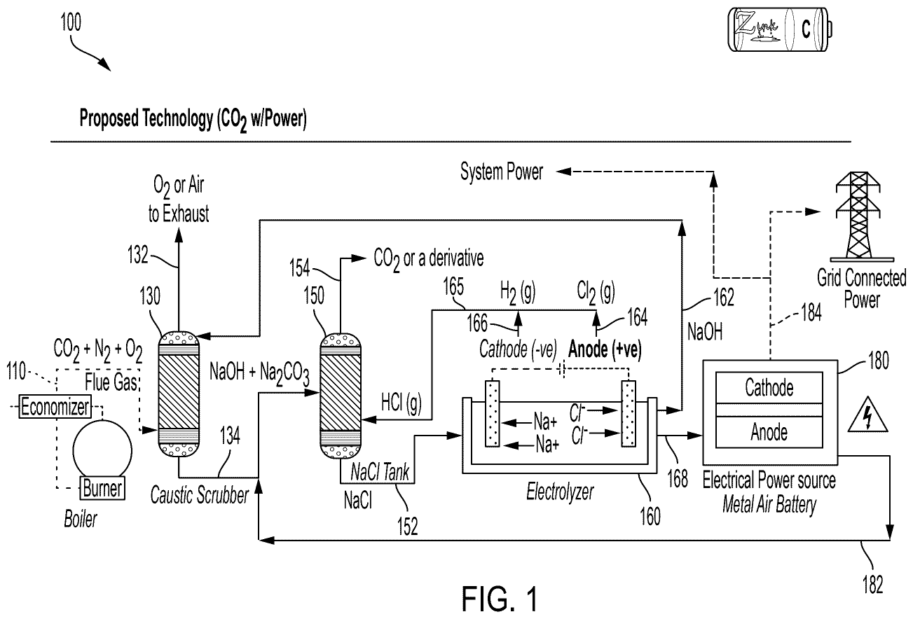

FIG. 1 illustrates an exemplary design of a carbon capture system 100 that is consistent with the present disclosure. The carbon capture system 100 is provided as an example to aid in describing the present technology. Although a particular arrangement of components is depicted in FIG. 1, the arrangement of such components should not be considered limiting of the present technology unless otherwise specified in the appended claims.

FIG. 1 illustrates a general system for the implementation of a carbon capture system 100. The carbon capture system 100 can include at least a caustic scrubber, which brings acidic gases from an industrial process, for example, into contact with a caustic solution, e.g., NaOH or KOH. The acidic gases then react chemically with the caustic solution, and are absorbed into the solution, removing the gases from the stream that comes into the caustic scrubber. The remaining gases that do not react with the caustic solution leave out of the top of the caustic scrubber, while the caustic solution, after the reaction, can be removed from the bottom of the scrubber.

The carbon capture system 100 can also include a neutralization scrubber 150. The neutralization scrubber 150, which brings the acidic gas, e.g., HCl, into contact with an alkaline scrubbing solution, e.g., NaOH and Na2CO3, removes the HCl gas from the scrubber. In one example, the HCl comes into contact with the NaOH and Na2CO3, which causes a chemical reaction that releases CO2 from the alkaline scrubbing solution and creates a salt, NaCl.

Carbon capture system 100 can also include an electrolyzer 160. The electrolyzer 160 can have a electrodes, e.g., nickel based compositions, in contact with an electrolyte to create electricity to drive chemical reactions by electrolysis. By creating a voltage difference between the electrodes, ion migration takes place and this can drive reduction and oxidation reactions at the electrodes. Electrolyzers can be useful in creating hydrogen gas that allows for the storage of electricity via chemical processes. Depending on the electrolyte, various reactions can take place; for example, if a brine or salt water-based solution is in the electrolyte, one possible reaction product is NaOH.

Carbon capture system 100 can also include an energy storage unit 180, which can be a metal air battery. The metal air battery can include a gas inlets that allow air into the system. When the energy storage unit is a metal air battery, it can also include an electrolyte to carry the charge created during operation. The electrolyte carries the charge from the anode to the cathode. The air can operate as the cathode for the energy storage unit 180 of FIG. 1. The metal air battery can include a current collector, a gas diffusion layer, and a passivating layer. The electrolyte is in contact with the electrodes and facilitates the chemical reaction driving electrical generation.

In operation, flue gases from an industrial source can be fed into the carbon capture system 100 as flue gas stream 110. The flue gases or off gases in flue gas stream 110 include CO₂ and other impurities can, and it can initially, be cooled via an interchanger or heat exchanger. The flue gases can be cooled to between 10 C and 70 C. If further cooling is needed, the intake process can include a secondary cooler. After cooling, the flue gases can be filtered by, for example, a coalescing filter to remove any oil from the flue gases. Finally, the flue gases can be fed into a particulate filter, which can be used to remove fine solids in the stream.

The heat that is removed from the input stream using the interchanger and the secondary cooler, can be reused within the system to heat other streams within the carbon capture process. For example, sometimes the caustic used in the process will need an elevated temperature and/or caustic that needs regeneration will need to be heated. By recycling the heat from the flue gases fed into system 100, the environmental impact of the system will be reduced and the economics of the plant will increase.

The flue gas stream 110 is then fed into the caustic scrubber 130, where the CO2 from the flue gas stream 110 is removed. In the caustic scrubber 130 the flue gases, including carbon dioxide, nitrogen and oxygen are contacted with a caustic solution, e.g., sodium hydroxide, potassium hydroxide, or calcium hydroxide, and the CO2 from the flue gas stream 110 is removed via a reaction with the caustic solution. In the caustic scrubber 130, when the flue gas stream 110 comes into contact with the caustic solution, a reaction occurs between the caustic solution and the carbon dioxide. The reaction will typically take the following form:

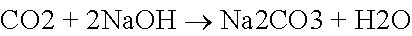

The majority of the carbon dioxide in the flue gases is captured in the caustic scrubber via the above reaction. The stream that includes NaOH, water, and Na2CO3 is diluted and referred to as a lean caustic stream 134 and exits the caustic scrubber 130 through the bottoms product. The distillate is substantially oxygen, nitrogen, and/or carbon monoxide. The distillate can either be further filtered and/or released into the atmosphere.

The lean caustic stream 134 from caustic scrubber 130 can be fed into a neutralization scrubber 150, where the NaOH and Na2CO3 is fed into the neutralization scrubber 150 and reacted with hydrochloric acid, to form NaCl and water along with carbon dioxide. The neutralization scrubber 150 is typically operated from about 20 C to slightly elevated temperature of 60 C. The remaining CO2 that is produced by the neutralization scrubber 150 can be released via CO2 outlet 154 to the environment or further processed as will be discussed with respect to later implementations of the present disclosure. The NaCl and water is transferred out via the neutralization scrubber bottoms 152 of the neutralization scrubber 150 to an electrolyzer 160 that can create an electrolyte for an energy storage unit 180 along with hydrogen gas and chlorine gas for the creation of the hydrochloric acid used in the neutralization scrubber 150.

The electrolyzer 160 receives the NaCl and water from the neutralization scrubber bottoms 152 and undergoes a chloralkali process to form hydrogen gas, chlorine gas and sodium hydroxide. The electrolyzer 160 passes a current through an aqueous solution that includes the NaCl from the neutralization scrubber 150, and electrolysis separates the ions and produces chlorine gas (Cl2) 164 through an oxidation reaction at the anode and hydrogen gas (H2) 166 and sodium hydroxide, through a reduction reaction, at the cathode. The electrolyzer 160 can include a membrane (not shown) to separate the products and facilitate the creation of the products, e.g., sodium hydroxide, hydrogen gas, and chlorine gas.

The hydrogen gas 166 and the chlorine gas 164 can be combined to form hydrochloric acid, HCl stream 165, that can be used in the neutralization scrubber 150 to facilitate formation of the carbon dioxide for CO2 outlet 154 and NaCl for the electrolyzer 160. The sodium hydroxide formed in the electrolyzer 160 can be fed via NaOH stream 162 into the caustic scrubber 130 and used to remove the carbon dioxide from the flue gases. The NaOH stream 162 can be chlorinated by combining it with the chlorine gas 164 from the electrolyzer 160, to additionally create NaOCl, NaCl, and water as a byproducts of the reactions. The chlorination reaction will typically be run at low temperatures, e.g., less than 25 C to prevent the hypochlorite product from decomposing. The NaOH can also be fed into an energy storage unit 180, via stream 168, where the energy storage unit 180 can utilize the NaOH as an electrolyte for power production in a metal air battery, which will be discussed in detail below. The energy storage unit 180 can be used to power the carbon capture system 100, to provide the power to the grid, or both. The NaOH from the energy storage unit 180 can be recycled into lean caustic stream 134 to improve the overall efficiency of carbon capture system 100.

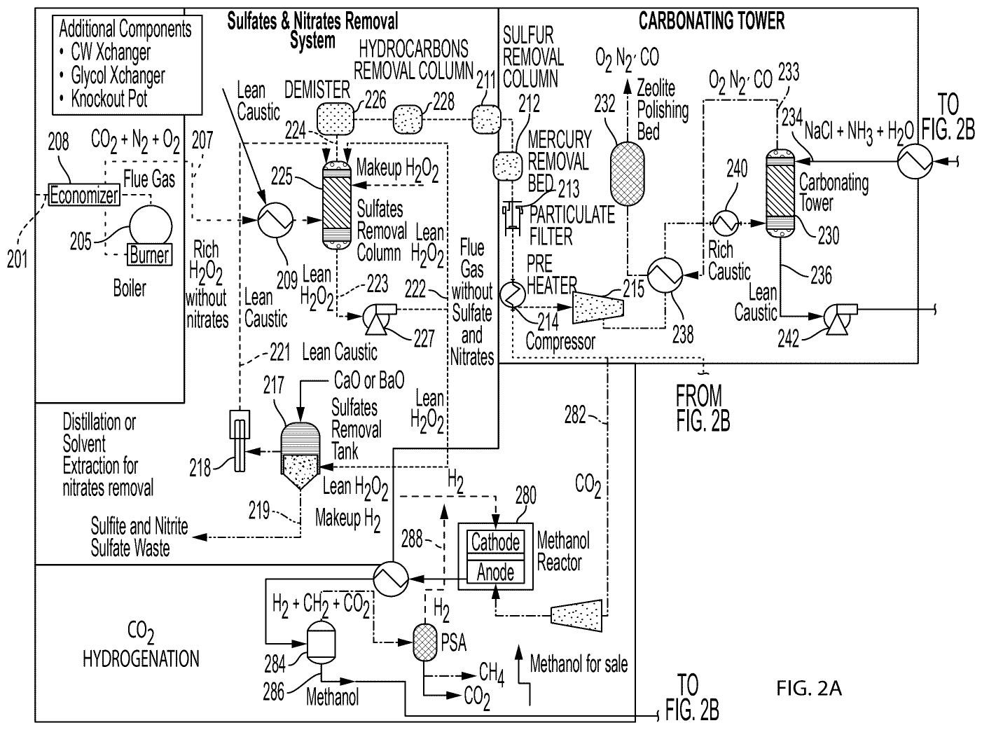

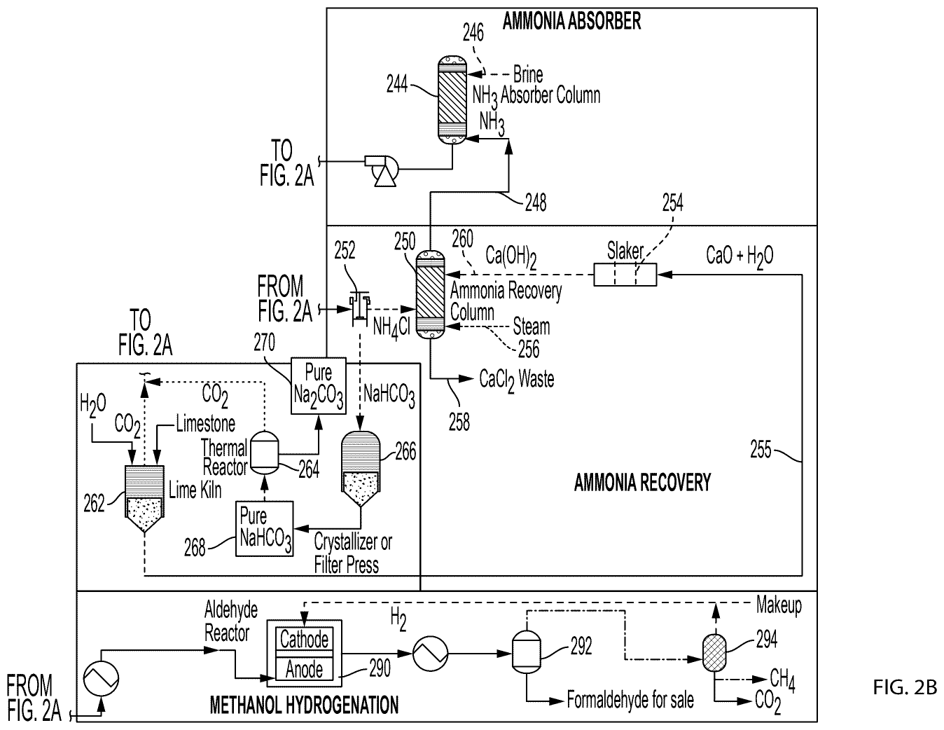

FIGS. 2A and 2B illustrate an exemplary carbon capture system 200 that uses a modified Solvay process production along with the carbon capture. The carbon capture system 200 includes a sulfate and nitrate removal system, a carbonating tower, and an ammonium recovery process. The carbon capture system 200 takes the flue gases from an industrial process and creates oxygen, nitrogen, carbon monoxide, sodium carbonate, soda ash, and recycles ammonia. The gases from the carbon capture system 200 meet the national standards for air quality and can be released. The sodium carbonate can be sold for use in industrial, commercial, or consumer applications. The carbon capture system 200 takes an environmentally problematic flue gases 201 and converts them to environmentally friendly products in an economic manner.

Carbon capture system 200, in addition to the caustic scrubber, that was discussed above with respect to FIG. 1, can also include a hydrogen peroxide scrubber 225. The hydrogen peroxide scrubber 225 of FIG. 2A works through the process of oxidation by the hydrogen peroxide of the compounds in the flue gases. For example, sulfur containing pollutants, e.g., SO₂ or H₂S, can be oxidized to form sulfuric acid and/or sulfate salts, depending on the operating conditions of the hydrogen peroxide scrubber 225. These sulfates can then be precipitated out of solution and removed from the system outside of the hydrogen peroxide scrubber 225. Similarly, the nitrates, which typically form from a combustion process, can be oxidized to form nitric acid and/or nitrate salts, depending on the operating conditions of the hydrogen peroxide scrubber 225. The nitrate salts can be soluble. The nitrates or nitric acid can be neutralized and then separated from solution outside the hydrogen peroxide scrubber 225.

Carbon capture system 200 can also include a demister 226. A demister removes the latent liquid droplets introduced to the stream during, e.g., a scrubbing process. The demister, therefore, can remove additional contaminants that remain as liquid droplets in a stream. Carbon capture system 200 can also include a hydrocarbon removal column 228, which can be, for example, a distillation column, an absorption column, an adsorption column, or similar. The hydrocarbon removal column 228 is capable of removing any remaining hydrocarbons that are present in the input stream. Generally, removal of hydrocarbons using distillation is sufficient to remove any hydrocarbon contaminants left in the stream, due to the differences in boiling point between the contaminants and the desired products, e.g., CO₂, O₂, and N₂. Carbon capture system 200 can also include a sulfur removal column 211. Sulfur removal column 211 can be, for example, an absorber/scrubber that uses reactive absorption of sulfur compounds by, for example, a zeolite, polymer resin, or an alkaline solution like limestone or lime. The reaction in the sulfur removal column 211 can remove substantially all of the remaining sulfur contaminants. Carbon capture system 200 can also include a mercury removal column 212. The mercury removal column 212 is typically going to comprise an adsorption column that includes, e.g., impregnated activated carbon, gold and/or silver on supports, or a zeolite tailored for mercury removal. The mercury removal column 212 can remove substantially all mercury that is present in an input stream. Additionally, carbon capture system 200 can include a particulate filter 213, which removes any particulates that may still be present in a stream that are larger than the size of the filter. Because the particulates will be considered quite large compared to the desired products, e.g., O₂, CO₂, and N₂, particulate filtering should remove all particulate that is present in the input stream.

As shown in FIG. 2A, the above-mentioned components can be configured in series, so that the output of the hydrogen peroxide scrubber 225 is transferred to the demister 226, then to the hydrocarbon removal column 228, then the top stream 224 is sent to sulfur removal column 211, followed by the mercury removal column 212, before finally being transferred to the particulate filter 213. However, the exact order of these components in series is not required by the present disclosure. Instead, the components can be in a different order, omitted, or placed in parallel as needed by the system. The carbon capture system 200 uses these series of components to create a purified stream into the carbonating tower 230, and when used as described herein below, it is possible to achieve a soda ash purity in excess of 95%, and most preferably in excess of 99% pure soda ash.

The components, e.g., hydrogen peroxide scrubber 225, demister 226, hydrocarbon removal column 228, sulfur removal column 211, mercury removal column 212, and particulate filter 213 are depicted as being after the hydrogen peroxide scrubber 225. However, it will be understood that, depending on the components within the flue gases 201, it can be preferable to put one or more of the components prior to the hydrogen peroxide scrubber 225. This can provide improved performance of the hydrogen peroxide scrubber 225 if the flue gases are particularly dirty. For example, if the flue gases contain a significant amount of fly ash, it would be preferable to place the particulate filter 213 prior to the hydrogen peroxide scrubber 225, so that the fly ash does not contaminate the hydrogen peroxide system.

In operation, carbon capture system 200 can receive flue gases 201 from an economizer 203, which allows for an exchange of heat between the flue gases 201 and the heated gas in stream 207. The flue gases 201 enter economizer 203 on the hot side and transfer energy to the heated gases in stream 207. The economizer 203 and the boiler 205 work together to efficiently heat gases in stream 207. The gases leave economizer 203 and enter boiler 205, where the gases are further heated and sent through the cold side of economizer 203, where additional heating takes place. After further heating by economizer 203, the heated gases in stream 207, which include carbon dioxide, nitrogen, and oxygen, enter flue gas interchanger 209, where the heat from the heated gases in stream 207 is used to heat the lean caustic stream 233 that is formed later in the process. s

After the flue gas interchanger 209, the cooled gases are introduced into the sulfate and nitrate removal system, where the substantial majority of any sulfates or nitrates present in the flue gases 201 are removed prior to the carbon dioxide being removed from the stream. The cooled gases of stream 207 are fed into the hydrogen peroxide scrubber 225, which can remove the sulfates and nitrates present in the flue gas. The gases are preferably cooled below 50° C., and more preferably cooled to between 10° C. and 30° C. prior to being introduced to the hydrogen peroxide scrubber 225. If temperatures exceed 50° C., the hydrogen peroxide can dissociate, creating hydrogen gas, which is a fire hazard. After the chemical scrubbing takes place, the bottoms 223 of the hydrogen peroxide scrubber 225 will contain the substantial majority of all sulfates and nitrates, which can be further purified.

The bottoms 223 from the hydrogen peroxide scrubber 225, which are a lean peroxide solution, are fed to a pump 227, which either recycles the stream back to the hydrogen peroxide scrubber 225 via stream 222 or transfers the bottoms 223 to a sulfate removal tank 217. The sulfate removal tank 217 can take the bottoms 223 and precipitate out the sulfates from the lean hydrogen peroxide solution via the addition of, for example, calcium oxide or barium oxide. The calcium oxide or barium oxide will typically form calcium sulfate or barium sulfate, respectively, each of which is insoluble in solution. Therefore, the calcium sulfate or barium sulfate will be removed via the waste stream 219. The nitrates will typically be removed via a different process, via separation column 218. Separation column 218 can be a distillation column, ion-exchange, electrodialysis, reverse osmosis, or solvent extraction column. The rich hydrogen peroxide stream 221, after the sulfates and nitrates are removed, is recycled back into the hydrogen peroxide scrubber 225, for the removal of sulfates and nitrates within the scrubber. The carbon capture system 200 can also have additional hydrogen peroxide added to the hydrogen peroxide scrubber 225, to make up for any losses during the sulfate and nitrate removal.

Top stream 224 from the hydrogen peroxide scrubber 225 is then transferred to a demister 226, which removes the latent liquid droplets introduced during the scrubbing process from the top stream 224. Once the liquid droplets are removed, the top stream 224 can be further purified by removing any hydrocarbons that may be present in the top stream 224. Hydrocarbon removal can take place via a hydrocarbon removal column 228, which can be, for example, a distillation column that removes the hydrocarbons from top stream 224. After the hydrocarbon removal column 228, the top stream 224 can be transferred to sulfur removal column 211. The sulfur removal column is, for example, an absorber that uses reactive absorption of sulfur compounds so that there are essentially no sulfur compounds left in the top stream 224 after it exits the sulfur removal column 211. The top stream can be transferred from the sulfur removal column 211 to mercury removal column 212. The mercury removal column 212 is typically going to comprise an adsorption column that includes, e.g., impregnated activated carbon. After removing essentially all of the mercury, the top stream 224 can be transferred to particulate filter 213, which removes any particulates that may still be present in the top stream 224.

Once the top stream 224 has been purified the top stream 224, essentially free from contaminants, needs to be prepared for CO₂ capture in carbonating tower 230. After the purification process, the stream is greater than 99.9% CO₂, CO, O₂, and N₂, with less than 50 ppm of sulfates and nitrates. By purifying the flue gases to remove the impurities, it is possible to create pure soda ash and/or sodium bicarbonate from the bottoms of the carbonating tower based on CO₂ from flue gas. Initially, the top stream 224 is heated by pre-heater 214 to between 30° C. and 70° C., with 32° C. being the preferred temperature. Pre-heater 214 can be any known vapor heating method, for example, a direct or indirect heater, which can be electric, gas, steam, etc., that can heat the vapor stream. After being heated by pre-heater 214, the top stream 224 is compressed by compressor 215 to manage deposition of any trace elemental sulfur present in the top stream 224. The compressed top stream 224 is then cooled in an interchanger 231, which can be an interchanger that cools the top stream 224 using the off gases from the carbonating tower 230. After the interchanger, the top stream 224 is fed into the carbonating tower 230 where the carbon dioxide is recovered.

Carbon capture system 200 can further include a carbonating tower 230, which can receive the purified and conditioned top stream 224 after the previous treatment stages, if implemented. The carbonating tower 230 also receives the ammonia input stream 234 from the ammonia absorber 244. Within the carbonating tower 230, carbon dioxide contained in the top stream 224 reacts with the ammoniated brine to form sodium bicarbonate and ammonium chloride. Sodium bicarbonate can precipitate within the carbonating tower 230 while ammonium chloride remains dissolved in solution, thereby generating a bottoms stream 236 that contains sodium bicarbonate solids in an ammonium chloride solution.

In certain embodiments, the carbonating tower 230 is operated to contact the purified flue gas (primarily comprising CO₂, N₂, and O₂) with an ammoniated brine (and, in some embodiments, a weak base such as a primary amine) under conditions that promote selective precipitation of sodium bicarbonate (NaHCO₃) while retaining ammonium chloride (NH₄Cl) and optional amine salts in solution. The carbonating tower 230 can operate at about 25° C. to about 50° C., preferably about 30° C. to about 40° C., and in one embodiment about 32° C. Lower temperatures favor NaHCO₃ supersaturation and crystal growth while limiting NH₃ loss. The carbonating tower 230 can operate at about 1 bar to about 3 bar, preferably about 1-2 bar. The brine entering through the ammonia input stream 234 of FIG. 2A can include a near-saturated NaCl brine (e.g., about 20-26 wt % NaCl) containing dissolved NH₃ (e.g., about 1-8 wt % NH₃ in the liquid phase). An amine and/or a weak base (e.g., methylamine) can be present at about 0.1-5 wt %, with the feed pH to tower 230 of greater than about 10 and more preferably above about 10.33 to yield the formation of Na₂CO₃. When an embodiment includes a weak acid, the feed pH of the brine solution can be adjusted to between about 6 to about 10.33, more preferably about 6.33 to about 10.33, to yield formation of NaHCO₃. By using either a weak acid or a weak base the system can be altered to form the desired product, either Na₂CO₃ or NaHCO₃.

The purified carbonating tower overhead product 233 from carbonating tower 230 can be passed through an exchanger 238 and a zeolite polishing bed 232, which is capable of removing trace contaminants such as hydrocarbons, sulfur, and mercury. The zeolite polishing bed 232 therefore produces a gaseous stream containing predominantly oxygen, nitrogen, and carbon monoxide, which may be vented, stored, or directed for other industrial uses.

The bottoms 236 from carbonating tower 230 can be sent, using pumps 242, to an ammonia recovery system. Initially, bottoms 236 are separated, via separator 252, into their components, which include ammonium chloride, NH₄Cl, which is sent to ammonia recovery column 250 and sodium bicarbonate, NaHCO₃, which is sent to crystallizer 266. Alternatively, the lean brine can also be fed to a plate and frame filter press or similar membrane filters to remove Total Suspended Solids (TSS). The wet cake from the filter press is recovered as sodium carbonate or sodium bicarbonate. The wet carbonate cake from the filter can be further dried using a dryer to remove residual moisture

Addressing the ammonia recovery first, the ammonia recovery column 250 receives the ammonium chloride, along with steam 256 and calcium hydroxide feed 260. In ammonia recovery column 250, the ammonium chloride solution is contacted with steam 256 and a calcium hydroxide feed 260. The calcium hydroxide for the calcium hydroxide feed 260 is provided from a slaker 254, which receives calcium oxide through a CaO inlet 255 and mixes it with water to produce the calcium hydroxide feed 260. Within the ammonia recovery column 250, ammonia is separated and recycled back to the ammonia absorber 244 and then provided to carbonating tower 230 through ammonia input stream 234. The ammonia recovery column 250 can be a distillation column that can operate at between about 80° C. to about 120° C., at pressures between about 1 bar to about 2 bar, and more preferably the ammonia recovery column 250 is operated at about 90° C. to about 110° C. at about 1 bar to about 2 bar. The bottoms of the ammonia recovery column 250 include a calcium chloride waste stream 258, which can be removed from the system.

The top of ammonia recovery column 250 is an ammonia feed 248, which can be provided to ammonia absorber 244. The ammonia absorber column 244, can receive a brine feed 246 in addition to the ammonia feed 248. The brine feed 246 comprises an aqueous sodium chloride solution, and the ammonia feed 248 is typically gaseous ammonia from the 250. Within the ammonia absorber column 244, the gaseous ammonia dissolves into the brine and reacts with water to form ammonium and hydroxide ions, thereby generating an alkaline ammoniated brine solution. The absorption of ammonia is an exothermic process, and therefore the ammonia absorber column 244 can include cooling jackets, intercoolers, or other temperature control features to maintain the absorption temperature within a preferred range of about 30° C. to about 50° C.

The ammonia absorber column 244 can be a packed column, tray column, or other mass-transfer device that maximizes contact between the ascending ammonia vapor and the descending brine liquid to achieve efficient absorption. The column is preferably operated at near-atmospheric pressure, e.g., between about 1 bar and about 2 bar, although slightly elevated pressures can be employed to improve ammonia capture efficiency and reduce ammonia slip.

The output from the bottom of the ammonia absorber column 244 is an ammoniated brine stream comprising sodium chloride, ammonia, and water. This ammoniated brine stream is then directed to the carbonating tower 230, where it is contacted with the conditioned top stream 224 to capture carbon dioxide and precipitate sodium bicarbonate.

The carbonate slurry from the bottoms 236, after separator 252, can be introduced to crystallizer 266, after separator 252 divides the stream into a solid fraction containing sodium bicarbonate and a liquid fraction containing ammonium chloride. The sodium bicarbonate 268 solids can be transferred to a crystallizer 266, where they are purified, and can be recovered as a high-purity sodium bicarbonate product NaHCO₃ 268, or further reacted in reactor 264, to form sodium carbonate, Na₂CO₃ 270. While crystallizer 266 is described, this stage of the process can also include a filter or other separation technique that can isolate the sodium bicarbonate. The reactor 264 can undertake a calcination stage, where the sodium bicarbonate is decomposed into the sodium carbonate, carbon dioxide, and water. This calcination process can take place at elevated temperatures of about 150° C. to about 200° C., with the reaction taking place at about 1 bar. The carbon dioxide from the reactor 264 can be recycled back into the top stream 224, where the carbon dioxide can be recaptured by the carbonating tower 230.

The Kiln 262 can receive limestone and water, to create calcium oxide, carbon dioxide, and water. The kiln can operate at about 700° C. to about 1100° C., preferably about 900° C. to about 1050° C., where the limestone, i.e., CaCO₃, is decomposed, to create a stream of calcium oxide and carbon dioxide. Kilns can operate with multiple stages as well. For example, the kiln 262 can have a preheating zone, which warms the limestone, and the preheating stage can operate at about 200 to about 800° C. Next, there can be a calcination zone, where CaCO3 is changed to CaO and CO2, and the calcination zone can operate between about 880 and about 1000° C. The calcination zone, in one example, aims to achieve a temperature of the core of the limestone of about 900° C. to about 950° C., where calcination can take place without overburning. Finally, there can be a third zone, a burning or hot zone that operates at about 1050° C. to about 1150° C., but is kept below about 1200° C. to avoid sintering. The lime is typically discharged at about 150 to about 250° C., and the exit gas can be around 300° C. to about 450° C. The exact calcination temperature will depend on partial pressure of the CO₂. A higher CO₂ partial pressure in the bed raises the target temperature. Further, if the kiln 262 has a good draft or the stones in the kiln are smaller, then the target temperature is lower. In one example, a rotary kiln can be used as kiln 262, and rotary kilns can target similar solids temperatures but will target higher flame and/or gas temperatures, e.g., about 1200° C. to about 1300° C.), still avoiding prolonged exposure that decreases reactivity.

The CaO inlet 255 transfers the calcium oxide and water to slaker 254, for use in the ammonia recovery column 250. The carbon dioxide produced by the decomposition of limestone in kiln 262, can be recycled into top stream 224, where the carbon dioxide can be recaptured by the carbonating tower 230. Further, as shown in FIG. 2B, the carbon dioxide from kiln 262 and the carbon dioxide from reactor 264 can be combined prior to being added to the top stream 224. Further, the carbon dioxide from either the kiln 262 or the reactor 264, or a combination from both, can be transferred to methanol reactor 280, via CO₂ inlet 282, as will be described in more detail below.

The captured CO₂ can either be pumped underground or converted into a derivative such as methanol, ethanol, aldehyde, adipic acid, catalytically converted to CO, arc plasma to convert to carbon black, or for use in bioluminescence, amongst other options. Furthermore, in another example, the captured CO₂ can be either captured or stored in a zeolite bed. The CO₂ that is captured in the zeolite bed can be recovered through the use of a caustic solution such as NaOH to form a concentrated stream of Na₂CO₃. The concentrated stream of Na₂CO₃ from the zeolite bed can be further recovered as soda ash through a vacuum crystallizer.

In one exemplary embodiment, the carbon capture system 200 of FIG. 2A can further include a methanol reactor 280, which is configured to receive a carbon dioxide stream through CO₂ inlet 282 and a hydrogen stream 288 generated by gas-liquid separator 284. The methanol reactor 280 can be an electrochemical hydrogenation reactor that includes an anode component, a cathode component, and an electrolyte configured to allow ion transport between the electrodes. The cathode of methanol reactor 280 can be coated with a catalyst, for example, copper, zinc oxide, alumina, palladium, ruthenium, or combinations thereof, that promotes the selective hydrogenation of CO₂ to methanol.

In operation, CO₂ introduced through CO₂ inlet 282 is reduced at the cathode in the presence of hydrogen supplied from gas-liquid separator 284 or hydrogen gas added to the system, producing methanol and water. At the anode, a reaction also takes place, e.g., water oxidation can occur, generating oxygen as a byproduct. The methanol reactor 280 can further include gas diffusion electrodes to improve mass transfer of CO₂ and H₂ to the catalyst sites, and in some embodiments, can be assisted by ultraviolet or infrared light to reduce the activation energy required for CO₂ hydrogenation.

The methanol reactor 280 can be operated at temperatures between about 50° C. and about 250° C., and at pressures between about 5 bar and about 50 bar, depending on system configuration. In certain embodiments, electrochemical operation allows the methanol reactor 280 to operate at lower pressures than conventional thermal catalytic methanol synthesis. The applied electrical potential across the electrodes of methanol reactor 280 can be between about 1.5 V and about 3.0 V, sufficient to drive the reduction of CO₂ at the cathode.

The output of methanol reactor 280 includes a methanol stream 286, which can be condensed and recovered for storage, sale, or downstream processing. In one embodiment, methanol stream 286 can be transferred to aldehyde reactor 290, where the methanol undergoes further hydrogenation and partial oxidation to produce formaldehyde. Byproduct oxygen generated at the anode of methanol reactor 280 can be vented, purified, or utilized elsewhere within system 200. Unreacted hydrogen and other off-gases can be separated and recycled by gas-liquid separator 284 and further purified by pressure swing adsorption, where hydrogen can be removed from the carbon dioxide and methane recovered from gas-liquid separator 284. The hydrogen that is recovered can be fed back into the methanol reactor 280 for use in the hydrogenation process.

Carbon capture system 200 of FIG. 2B can further include an aldehyde reactor 290, which is configured to receive the methanol stream 286 generated from methanol reactor 280. Within aldehyde reactor 290, the methanol is further hydrogenated and partially oxidized to form formaldehyde. The aldehyde reactor 290 can be an electrochemical reactor or a thermal catalytic reactor. In one embodiment, aldehyde reactor 290 comprises an anode component, a cathode component, and an electrolyte, with electrodes coated in catalysts such as silver, iron-molybdenum, copper, or vanadium-based formulations suitable for methanol-to-formaldehyde conversion. In another embodiment, aldehyde reactor 290 operates as a thermal catalytic reactor in the presence of oxygen and hydrogen, utilizing selective oxidation pathways to yield formaldehyde with minimal side products.

The aldehyde reactor 290 can be operated at a temperature between about 200° C. and about 450° C. and at a pressure between about 1 bar and about 10 bar, depending on the selected catalytic pathway. In electrochemical embodiments, the applied electrical potential across the electrodes of aldehyde reactor 290 can be sufficient to drive the hydrogenation-oxidation reaction of methanol to formaldehyde, while generating oxygen or hydrogen as secondary products.

The product stream from aldehyde reactor 290 can be directed to a formaldehyde tower 292, where the formaldehyde is condensed, separated, and recovered at high purity. Formaldehyde tower 292 can include fractionation trays, condensers, or absorption columns configured to isolate formaldehyde from unreacted methanol, water, and minor byproducts. The recovered formaldehyde can be stored, packaged, or transferred for industrial use, including polymer production, resins, adhesives, and chemical intermediates.

Carbon capture system 200 of FIG. 2B can also include a hydrogen tower 294 that is fluidly connected to aldehyde reactor 290. The hydrogen tower 294 is configured to separate unreacted hydrogen and other gaseous components from the aldehyde reactor off-gas stream. The recovered hydrogen can be recycled back to aldehyde reactor 290 or methanol reactor 280, improving overall hydrogen efficiency within the system. Off-gases that are not recycled may be vented, flared, or further treated depending on system requirements.

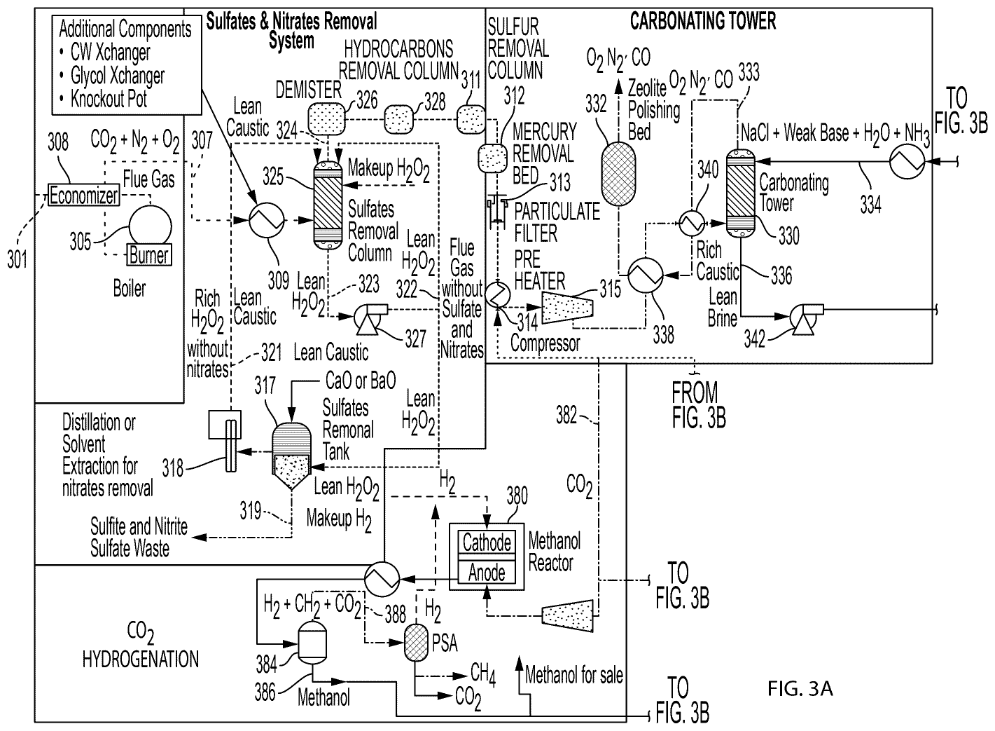

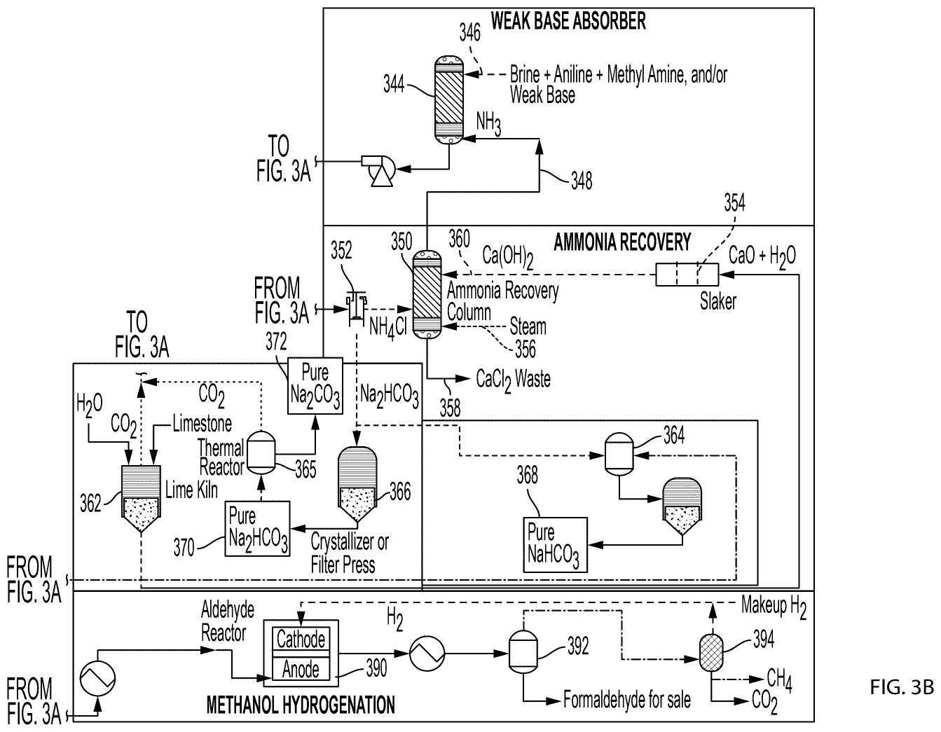

FIGS. 3A and 3B illustrate an exemplary carbon capture system 300 that uses a weak base along with the ammonia to facilitate carbon capture. The carbon capture system 300 includes a sulfate and nitrate removal system, a carbonating tower, ammonia recovery, and a sodium carbonate production system. The carbon capture system 300 takes the flue gases from an industrial process and creates oxygen, nitrogen, carbon monoxide, sodium carbonate, and soda ash. The gases from the carbon capture system 300 meet the national standards for air quality and are able to be released. The sodium carbonate can be sold for use in industrial, commercial, or consumer applications. The carbon capture system 300 takes an environmentally problematic flue gases 301 and converts them to environmentally friendly products in an economic manner. Similar components described in detail in FIG. 2A and FIG. 2B will operate in a similar manner for FIG. 3A and FIG. 3B, unless otherwise noted below.

Carbon capture system 300 can include a hydrogen peroxide scrubber 325. The hydrogen peroxide scrubber 325 of FIG. 3A works through the process of oxidation by the hydrogen peroxide of the compounds in the flue gases. For example, sulfur containing pollutants, e.g., SO₂ or H₂S, can be oxidized to form sulfuric acid and/or sulfate salts, depending on the operating conditions of the hydrogen peroxide scrubber 325. These sulfates can then be precipitated out of solution and removed from the system outside of the hydrogen peroxide scrubber 325. Similarly, the nitrates, which typically form from a combustion process, can be oxidized to form nitric acid and/or nitrate salts, depending on the operating conditions of the hydrogen peroxide scrubber 325. The nitrate salts can be soluble. The nitrates or nitric acid can be neutralized and then separated from solution outside the hydrogen peroxide scrubber 325.

Carbon capture system 300 of FIG. 3A can also include a demister 326. A demister removes the latent liquid droplets introduced to the stream during, e.g., a scrubbing process. The demister, therefore, can remove additional contaminates that remains as liquid droplets in a stream. Carbon capture system 300 can also include a hydrocarbon removal column 328, which can be, for example, a distillation column, an absorption column, an adsorption column, or similar. The hydrocarbon removal column 328 is capable of removing any remaining hydrocarbons that are present in the input stream. Generally, removal of hydrocarbons using distillation is sufficient to remove any hydrocarbon contaminants left in the stream, due to the differences in boiling point between the contaminants and the desired products, e.g., CO₂, O₂, and N₂. Carbon capture system 300 can also include a sulfur removal column 311. Sulfur removal column 311 can be, for example, an absorber/scrubber that uses reactive absorption of sulfur compounds by for example, a zeolite, polymer resin, or an alkaline solution like limestone or lime. The reaction in the sulfur removal column 311 can remove substantially all of the remaining sulfur contaminants. Carbon capture system 300 can also include a mercury removal column 312. The mercury removal column 312 is typically going to comprise an adsorption column that includes, e.g., impregnated activated carbon, gold and/or silver on supports, or a zeolite tailored for mercury removal. The mercury removal column 312 can remove substantially all mercury that is present in an input stream. Additionally, carbon capture system 300 can include a particulate filter 313 which removes any particulates that may still be present in a stream that are lager than the size of the filter. Because the particulates will be considered quite large compared to the desired products, e.g., O₂, CO₂ and N₂, particulate filtering should remove all particulate that is present in the input stream.

As shown in FIG. 3A, the above mentioned components can be configured in series, so that the output of the hydrogen peroxide scrubber 325 is transferred to the demister 326, then to the hydrocarbon removal column 328, then the top stream 324 is sent to sulfur removal column 311, followed by the mercury removal column 312, before finally being transferred to the particulate filter 313. However, the exact order of these components in series is not required by the present disclosure. Instead, the components can be in a different order, omitted, or placed in parallel as needed by the system. The carbon capture system 300 uses these series of components to create a purified stream into the carbonating tower 330.

The components, e.g., hydrogen peroxide scrubber 325, demister 326, hydrocarbon removal column 328, sulfur removal column 311, mercury removal column 312, and particulate filter 313 are depicted as being after the hydrogen peroxide scrubber 325. However, it will be understood that, depending on the components within the flue gases 301, it can be preferable to put one or more of the components prior to the hydrogen peroxide scrubber 325. This can provide improved performance of the hydrogen peroxide scrubber 325 if the flue gases are particularly dirty. For example, if the flue gases contain a significant amount of fly ash, it would be preferable to place the particulate filter 313 prior to the hydrogen peroxide scrubber 325, so that the fly ash does not contaminate the hydrogen peroxide system.

In operation, carbon capture system 300 can receive flue gases 301 from an economizer 308, which allows for an exchange of heat between the flue gases 301 and the heated gas in stream 307. The flue gases 301 enter economizer 308 on the hot side and transfer energy to the heated gases in stream 307. The economizer 308 and the boiler 305 work together to efficiently heat gases in stream 307. The gases leave economizer 308 and enter boiler 305, where the gases are further heated and sent through the cold side of economizer 308, where additional heating takes place. After further heating by economizer 308, the heated gases in stream 307, which include carbon dioxide, nitrogen, and oxygen, enter cooler 309, where the heat from the heated gases in stream 307 are removed.

After the cooler 309, the cooled gases are introduced into the sulfate and nitrate removal system, where the substantial majority of any sulfates or nitrates present in the flue gases 301 are removed prior to the carbon dioxide being removed from the stream. The cooled gases of stream 307 are fed into the hydrogen peroxide scrubber 325, which can remove the sulfates and nitrates present in the flue gas. The gases are preferably cooled below 50° C., and more preferably cooled to between 10° C. and 30° C. prior to being introduced to the hydrogen peroxide scrubber 325. If temperatures exceed 50° C., the hydrogen peroxide can dissociate, creating hydrogen gas, which is a fire hazard. After the chemical scrubbing takes place, the bottoms 323 of the hydrogen peroxide scrubber 325 will contain the substantial majority of all sulfates and nitrates, which can be further purified.

The bottoms 323 from the hydrogen peroxide scrubber 325, which are a lean peroxide solution, are fed to a pump 327 which either recycles the stream back to the hydrogen peroxide scrubber 325 via stream 322 or transfers the bottoms 323 to a sulfate removal tank 317. The sulfate removal tank 317 can take the bottoms 323 and precipitate out the sulfates from the lean hydrogen peroxide solution via the addition of, for example, calcium oxide or barium oxide. The calcium oxide or barium oxide will typically form calcium sulfate or barium sulfate, respectively, each of which is insoluble in solution. Therefore, the calcium sulfate or barium sulfate will be removed via the waste stream 319. The nitrates will typically be removed via a different process, via separation column 318. Separation column 318 can be a distillation column, ion-exchange, electrodialysis, reverse osmosis, or solvent extraction column. The rich hydrogen peroxide stream 321, after the sulfates and nitrates are removed, is recycled back into the hydrogen peroxide scrubber 325, for the removal of sulfates and nitrates within the scrubber. The carbon capture system 300 can also have additional hydrogen peroxide added to the hydrogen peroxide scrubber 325, to make up for any losses during the sulfate and nitrate removal.

Top stream 324 from the hydrogen peroxide scrubber 325 is then transferred to a demister 326, which removes the latent liquid droplets introduced during the scrubbing process from the top stream 324. Once the liquid droplets are removed, the top stream 324 can be further purified by removing any hydrocarbons that may be present in the top stream 324. Hydrocarbon removal can take place via a hydrocarbon removal column 328, which can be, for example, a distillation column, that removes the hydrocarbons from top stream 324. After the hydrocarbon removal column 328, the top stream 324 can be transferred to sulfur removal column 311. The sulfur removal column is, for example, an absorber that uses reactive absorption of sulfur compounds so that there are essentially no sulfur compounds left in the top stream 324 after it exits the sulfur removal column 311. The top stream can be transferred from the sulfur removal column 311 to mercury removal column 312. The mercury removal column 312 is typically going to comprise an adsorption column that includes, e.g., impregnated activated carbon. After removing essentially all of the mercury, the top stream 324 can be transferred to particulate filter 313, which removes any particulates that may still be present in the top stream 324.

Once the top stream 324 has been purified the top stream 324, essentially free from contaminants, needs to be prepared for CO₂ capture in carbonating tower 330. Initially, the top stream 324 is heated by pre-heater 314 to between 30° C. to 70° C., with 32° C. being the preferred temperature. Pre-heater 314 can be any known vapor heating method, for example, a direct or indirect heater, which can be electric, gas, steam, etc., that can heat the vapor stream. After being heated by pre-heater 314, the top stream 324 is compressed by compressor 315 to manage deposition of any trace elemental sulfur present in the top stream 324. The compressed top stream 324 is then cooled in a interchanger 331, which can be an interchanger that cools the top stream 324 using the off gases from carbonating tower 330. After the interchanger, the top stream 324 is fed into the carbonating tower 330 where the carbon dioxide is recovered.

Carbon capture system 300 of FIG. 3A can further include a carbonating tower 330, which is can receive the purified and conditioned top stream 324 after the previous treatment stages, if implemented. The carbonating tower 330 also receives the ammonia input stream 334 from the weak base absorber column 344. Within the carbonating tower 330, carbon dioxide contained in the top stream 324 reacts with the ammoniated brine to form sodium bicarbonate and ammonium chloride. Sodium bicarbonate can precipitate within the carbonating tower 330 while ammonium chloride remains dissolved in solution, thereby generating a bottoms stream 336 that contains sodium bicarbonate solids in an ammonium chloride solution.

The purified carbonating tower overhead product 333 from carbonating tower 330 can be passed through an exchanger 338 and a zeolite polishing bed 332, which is capable of removing trace contaminants such as hydrocarbons, sulfur, and mercury. The zeolite polishing bed 332 therefore produces a gaseous stream containing predominantly oxygen, nitrogen, and carbon monoxide, which may be vented, stored, or directed for other industrial uses.

The bottoms 336 from carbonating tower 330 can be sent, using pumps 342, to an ammonia recovery system. Initially, bottoms 336 are separated, via separator 352, into its components, which include ammonium chloride, NH4Cl, which is sent to ammonia recovery column 350 and soda ash, Na₂CO₃, which is sent to crystallizer 366 or reactor 364.

Addressing the ammonia recovery first, the ammonia recovery column 350 receives the ammonium chloride, along with steam 356 and calcium hydroxide feed 360. In ammonia recovery column 350, the ammonium chloride solution is contacted with steam 356 and a calcium hydroxide feed 360. The calcium hydroxide for the calcium hydroxide feed 360 is provided from a slaker 354, which receives calcium oxide through a CaO inlet 355 and mixes it with water to produce the calcium hydroxide feed 360. Within the ammonia recovery column 350, ammonia is separated and recycled back to the weak base absorber column 344 and then provided to carbonating tower 330 through ammonia input stream 334. The ammonia recovery column 350 can be a distillation column that can operate at between about 80° C. to about 120° C., at pressures between about 1 bar to about 2 bar, and more preferably the ammonia recovery column 350 is operated at about 90° C. to about 110° C. at about 1 bar to about 2 bar. The bottoms of the ammonia recovery column 350 include a calcium chloride waste stream 358, which can be removed from the system.

The top of ammonia recovery column 350 is an ammonia feed 348, which can be provided to weak base absorber column 344. The weak base absorber column 344, can receive a brine feed 346 in addition to the ammonia feed 348. The weak base feed 346 comprises an aqueous sodium chloride solution along with at least one of aniline, methyl amine, and/or a weak base, and the ammonia feed 348 is typically gaseous ammonia from the 350. Within the weak base absorber column 344, the gaseous ammonia dissolves into the brine and reacts with water to form ammonium and hydroxide ions, thereby generating an alkaline ammoniated brine solution. The presence of at least one of aniline, methyl amine, and/or a weak base in the brine feed enhances the alkalinity of the absorbent solution. For example, when methyl amine is used, it can dissolve in water to form methylammonium (CH₃NH₃) and hydroxide ions. Weak base absorber column 344 therefore generates an ammoniated and aminated brine stream comprising sodium chloride, ammonia, methylamine, and water. The absorption of ammonia and amine is exothermic, and thus weak base absorber column 344 can include cooling jackets or intercoolers to maintain the operating temperature between about 30° C. and about 50° C.

The ammonia absorber column 344 can be a packed column, tray column, or other mass-transfer device that maximizes contact between the ascending ammonia vapor and the descending brine liquid to achieve efficient absorption. The column is preferably operated at near-atmospheric pressure, e.g., between about 1 bar and about 2 bar, although slightly elevated pressures can be employed to improve ammonia capture efficiency and reduce ammonia slip.

The output from the bottom of the weak base absorber column 344 is an ammoniated brine stream comprising sodium chloride, ammonia, and water. This ammonia input stream 334 is then directed to the carbonating tower 330, where it is contacted with the conditioned top stream 324 to capture carbon dioxide and precipitate sodium bicarbonate and can also include methylammonium bicarbonate due to the addition of methyl amine to the ammonia input stream 334.

The carbonate slurry from the bottoms 336, after separator 352, can be introduced to reactor 364 and/or crystallizer 366, after separator 352 divides the stream into a solid fraction containing soda ash and a liquid fraction containing ammonium chloride. The soda ash solids can be transferred to a crystallizer 366, where they are purified, and can be recovered as a high-purity soda ash product Na₂CO₃ 370. The soda ash product in 370 can include up to about 15% water, which is an acceptable proportion for the product. However, some uses of soda ash rely on a more pure product. Accordingly, the soda ash from 370 can be dried further in thermal reactor 365 where the amount of water is reduced to about less than 99%, depending on the product quality required. Thermal reactor 365 can be used to dry the soda ash to any portion between 15% water and essentially dry at less than 1% water. While crystallizer 366 is described, this stage of the process can also include a filter or other separation technique that can isolate the sodium carbonate. The soda ash solids can additionally or alternatively be further reacted in reactor 364, optionally including the addition of water and/or a weak acid, to form sodium bicarbonate, NaHCO₃ 368. The sodium carbonate added to reactor 364 can produce the sodium bicarbonate through a carbonation process. In this embodiment, sodium carbonate is contacted with a stream of carbon dioxide in the presence of water under controlled conditions. The sodium carbonate dissolves in the aqueous phase, and the introduction of carbon dioxide results in the formation of carbonic acid, which protonates the carbonate anion to yield sodium bicarbonate. The carbonation process can take place in reactor 364 at a temperature between about 30° C. and about 60° C., and at pressures from about 1 bar to about 5 bar, to promote dissolution and carbonation without decomposition. The sodium bicarbonate formed in this manner can be crystallized or otherwise separated from the aqueous medium to yield a high-purity sodium bicarbonate product 368, which can be used directly or further processed within the system.

The kiln 362 can receive limestone and water, to create calcium oxide, carbon dioxide, and water. The kiln 362 can receive limestone and water, to create calcium oxide, carbon dioxide, and water. The kiln can operate at about 700° C. to about 1100° C., preferably about 900° C. to about 1050° C., where the limestone, i.e., CaCO₃, is decomposed, to create a stream of calcium oxide and carbon dioxide. Kilns can operate with multiple stages as well. For example, the kiln 362 can have a preheating zone, which warms the limestone, and the preheating stage can operate at about 200 to about 800° C. Next, there can be a calcination zone, where CaCO3 is changed to CaO and CO2, and the calcination zone can operate between about 880 and about 1000° C. The calcination zone, in one example, aims to achieve a temperature of the core of the limestone of about 900 to about 950° C., where calcination can take place without overburning. Finally, there can be a third zone, a burning or hot zone that operates at about 1050° C. to about 1150° C., but is kept below about 1200° C. to avoid sintering. The lime is typically discharged at about 150° C. to about 250° C., and the exit gas can be around 300° C. to about 450° C. The exact calcination temperature will depend on partial pressure of the CO₂. A higher CO₂ partial pressure in the bed raises the target temperature. Further, if the kiln 262 has a good draft or the stones in the kiln are smaller, then the target temperature is lower. In one example, a rotary kiln can be used as kiln 262, and rotary kilns can target similar solids temperatures but will target higher flame and/or gas temperatures, e.g., about 1200° C. to about 1300° C.), still avoiding prolonged exposure that decreases reactivity.

The CaO inlet 355 transfers the calcium oxide and water to slaker 354, for use in the ammonia recovery column 350. The carbon dioxide produced by the decomposition of limestone in kiln 362, can be recycled into top stream 324, where the carbon dioxide can be recaptured by the carbonating tower 330. Further, as shown in FIG. 3A, the carbon dioxide from kiln 362 and the carbon dioxide from reactor 364 can be combined prior to being added to the top stream 324. Further, the carbon dioxide from either the kiln 362 or the reactor 364, or a combination from both, can be transferred to methanol reactor 380, via CO₂ inlet 382 or to reactor 364, as will be described in more detail below.

Carbon capture system 300 of FIG. 3A can further include a methanol reactor 380, which is configured to receive a carbon dioxide stream through CO₂ inlet 382 and a hydrogen stream 388 generated by gas-liquid separator 384. The methanol reactor 380 can be an electrochemical hydrogenation reactor that includes an anode component, a cathode component, and an electrolyte configured to allow ion transport between the electrodes. The cathode of methanol reactor 380 can be coated with a catalyst, for example, copper, zinc oxide, alumina, palladium, ruthenium, or combinations thereof, that promotes the selective hydrogenation of CO₂ to methanol.

In operation, CO₂ introduced through CO₂ inlet 382 is reduced at the cathode in the presence of hydrogen supplied from gas-liquid separator 384 or hydrogen gas added to the system, producing methanol and water. At the anode, a reaction also takes place, e.g., water oxidation can occur, generating oxygen as a byproduct. The methanol reactor 380 can further include gas diffusion electrodes to improve mass transfer of CO₂ and H₂ to the catalyst sites, and in some embodiments, can be assisted by ultraviolet or infrared light to reduce the activation energy required for CO₂ hydrogenation.

The methanol reactor 380 can be operated at temperatures between about 50° C. and about 250° C., and at pressures between about 5 bar and about 50 bar, depending on system configuration. In certain embodiments, electrochemical operation allows the methanol reactor 380 to operate at lower pressures than conventional thermal catalytic methanol synthesis. The applied electrical potential across the electrodes of methanol reactor 380 can be between about 1.5 V and about 3.0 V, sufficient to drive the reduction of CO₂ at the cathode.

The output of methanol reactor 380 of FIG. 3A includes a methanol stream 386, which can be condensed and recovered for storage, sale, or downstream processing. In one embodiment, methanol stream 386 can be transferred to aldehyde reactor 390 of FIG. 3B, where the methanol undergoes further hydrogenation and partial oxidation to produce formaldehyde. Byproduct oxygen generated at the anode of methanol reactor 380 can be vented, purified, or utilized elsewhere within system 300. Unreacted hydrogen and other off-gases can be separated and recycled by gas-liquid separator 384 and further purified by pressure swing adsorption, where hydrogen can be removed from the carbon dioxide and methane recovered from gas-liquid separator 384. The hydrogen that is recovered can be fed back into the methanol reactor 380 for use in the hydrogenation process.

Carbon capture system 300 of FIG. 3B can further include an aldehyde reactor 390, which is configured to receive the methanol stream 386 generated from methanol reactor 380. Within aldehyde reactor 390, the methanol is further hydrogenated and partially oxidized to form formaldehyde. The aldehyde reactor 390 can be an electrochemical reactor or a thermal catalytic reactor. In one embodiment, aldehyde reactor 390 comprises an anode component, a cathode component, and an electrolyte, with electrodes coated in catalysts such as silver, iron-molybdenum, copper, or vanadium-based formulations suitable for methanol-to-formaldehyde conversion. In another embodiment, aldehyde reactor 390 operates as a thermal catalytic reactor in the presence of oxygen and hydrogen, utilizing selective oxidation pathways to yield formaldehyde with minimal side products.

The aldehyde reactor 390 can be operated at a temperature between about 200° C. and about 450° C. and at a pressure between about 1 bar and about 10 bar, depending on the selected catalytic pathway. In electrochemical embodiments, the applied electrical potential across the electrodes of aldehyde reactor 390 can be sufficient to drive the hydrogenation-oxidation reaction of methanol to formaldehyde, while generating oxygen or hydrogen as secondary products.

The product stream from aldehyde reactor 390 can be directed to a formaldehyde tower 392, where the formaldehyde is condensed, separated, and recovered at high purity. Formaldehyde tower 392 can include fractionation trays, condensers, or absorption columns configured to isolate formaldehyde from unreacted methanol, water, and minor byproducts. The recovered formaldehyde can be stored, packaged, or transferred for industrial use, including polymer production, resins, adhesives, and chemical intermediates.

Carbon capture system 300 of FIG. 3B can also include a hydrogen tower 394 that is fluidly connected to aldehyde reactor 390. The hydrogen tower 394 is configured to separate unreacted hydrogen and other gaseous components from the aldehyde reactor off-gas stream. The recovered hydrogen can be recycled back to aldehyde reactor 390 or methanol reactor 380, improving overall hydrogen efficiency within the system. Off-gases that are not recycled may be vented, flared, or further treated depending on system requirements.

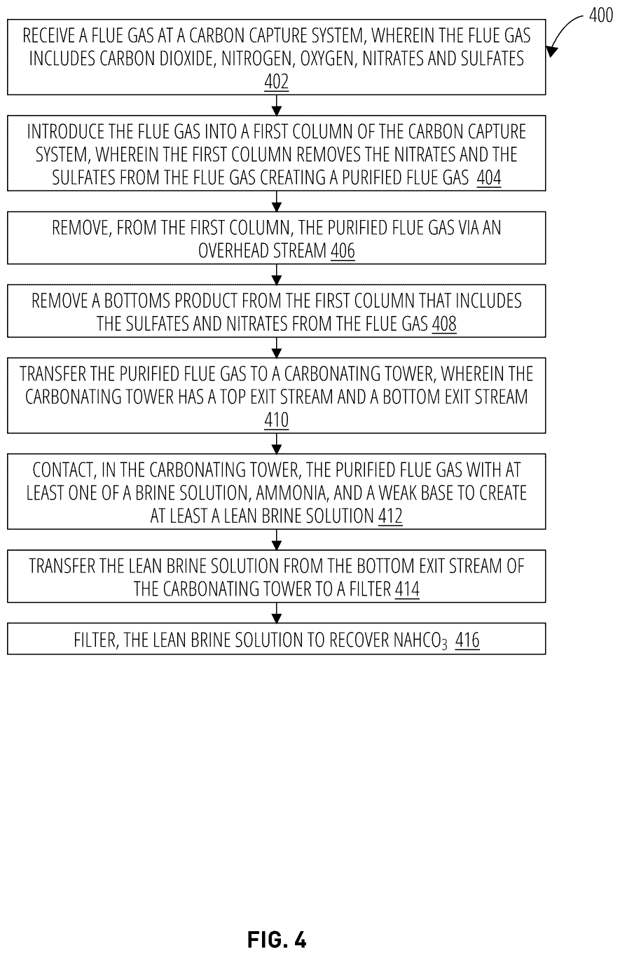

FIG. 4 illustrates an example method of using the carbon dioxide from flue gases to make soda ash or sodium bicarbonate. Although the example method depicts a particular sequence of operations, the sequence may be altered without departing from the scope of the present disclosure. For example, some of the operations depicted may be performed in parallel, may be excluded or added, or may be performed in a different sequence that does not materially affect the function of the method. In other examples, different components of an example device or system that implements the method may perform functions at substantially the same time or in a specific sequence.

In block 402, routine 400 receives a flue gas at a carbon capture system, wherein the flue gas includes carbon dioxide, nitrogen, oxygen, nitrates and sulfates. In some embodiments, the flue gas further includes moisture, hydrocarbons, sulfur compounds (e.g., SO₂, SO₃, H₂S), mercury, and particulates. The flue gas can be thermally conditioned via an economizer and/or interchangers to a temperature ≤50° C., preferably 10-30° C., to protect oxidants used downstream and to improve gas-liquid mass transfer.

In block 404, routine 400 introduces the flue gas into a first column of the carbon capture system, wherein the first column removes the nitrates and the sulfates from the flue gas creating a purified flue gas. The first column is configured to remove nitrates and sulfates. In one embodiment, the first column is a hydrogen peroxide scrubber operated as an oxidation absorber, wherein hydrogen peroxide oxidizes nitrogen- and sulfur-containing species to nitric acid/nitrate and sulfuric acid/sulfate species in the liquid phase. The column can include spray nozzles and/or structured packing to maximize gas-liquid interfacial area, and is preferably operated below 50° C. (more preferably 10-30° C.) to limit peroxide decomposition and hydrogen evolution.

In block 406, routine 400 removes, from the first column, the purified flue gas via an overhead stream. The overhead stream can optionally pass through a demister to remove entrained droplets; a hydrocarbon removal column (distillation/absorption/adsorption) to reduce volatile organics; a sulfur removal column to remove residual sulfur species; a mercury removal column (e.g., impregnated activated carbon or tailored zeolite); and/or a particulate filter, thereby yielding a gas stream predominantly comprising CO₂, N₂, and O₂ suitable for carbonation.

In block 408, routine 400 removes a bottoms product from the first column that includes the sulfates and nitrates from the flue gas. The bottoms can be directed to a sulfate/nitrate workup, wherein sulfates are precipitated as CaSO₄ or BaSO₄ by addition of alkaline-earth reagents, and nitrates are separated and/or neutralized via distillation, ion exchange, electrodialysis, reverse osmosis, or solvent extraction. The bottoms product, with the contaminants removed, can be recycled back into the first column.

In block 410, routine 400 transfers the purified flue gas to a carbonating tower, wherein the carbonating tower has a top exit stream and a bottom exit stream. Prior to entering carbonating tower 230 or carbonating tower 330, the gas can be preheated to (e.g., 30-70° C., preferably about 30° C. to about 35° C.) and/or compressed to manage deposition of trace species.

In block 412, routine 400 contacts, in the carbonating tower, the purified flue gas with at least one of a brine solution, ammonia, and a weak base to create at least a lean brine solution. In one embodiment, NH₃ absorber 244 supplies an ammoniated brine (formed by contacting brine feed 246 with NH₃ feed 248 at about 30-50° C. and ˜1-2 bar), which upon contact with CO₂ in carbonating tower 230 of FIG. 2 or carbonating tower 330 of FIG. 3 forms sodium bicarbonate (NaHCO₃) or Soda Ash (Na₂CO₃), respectively, that precipitate out, while ammonium chloride (NH₄Cl) remains dissolved. In another embodiment, the contacting liquid further includes a weak base such as a primary amine (e.g., methylamine) to increase CO₂ loading as amine-bicarbonate/carbamate species; operating parameters (temperature, NH₃/amine ratio, ionic strength, residence time) are selected to maintain preferential NaHCO₃ precipitation while retaining ammonium and/or amine salts in solution. Carbonating tower 230 or Carbonating tower 330 can utilize trays or structured packing, antifoam management, and demisting at the top outlet.

In block 414, routine 400 transfer the lean brine solution from the bottom exit stream of the carbonating tower to a filter. The bottoms slurry comprising NaHCO₃ solids suspended in an NH₄Cl-rich solution and, when present, amine salts from the bottom exit of carbonating tower 230 are filtered by a filter, e.g., separator 252 of FIG. 2 or separator 352 of FIG. 3. The slurry may be cooled to increase solids fraction, thickened, and pumped to solid-liquid separation (e.g., rotary vacuum drum filter, filter press, or centrifuge), with optional cold-wash to reduce chloride inclusion in the cake

In block 416, routine 400 filters, the lean brine solution to recover NaHCO₃. The filtration yields a NaHCO₃ solid product (optionally directed to crystallizer 266 for purity and crystal size control) and a filtrate (solution). Filtering the lean brine solution also recovers NH₄Cl in the filtrate as a concentrated NH₄Cl stream suitable for ammonia recovery.

Furthermore, after the lean brine solution is filtered in block 416, in some embodiments, routine 400 can further include transferring the NaHCO₃ recovered in block 416 to a reactor, e.g., reactor 264 and calcining the NaHCO₃ at elevated temperature (e.g., 150-200° C., ˜0.8-1.2 bar) to form substantially pure Na₂CO₃ and to release CO₂ and H₂O vapor. The solid Na₂CO₃ 270 can be used downstream as a commercial soda-ash product. The off-gas from the calcination process is collected as a CO₂-rich stream that can be used later in the process to create further products, e.g., methanol or formaldehyde.

In certain embodiments, routine 400 further includes transferring the CO₂ released during calcination to a reactor, e.g., methanol reactor 280 via CO₂ inlet 282 or methanol reactor 380 via CO₂ inlet 382, and hydrogenating the CO₂ to produce methanol. Methanol reactor 280 or methanol reactor 380 can be an electrochemical hydrogenation reactor (anode, cathode, electrolyte, gas-diffusion electrodes) or a thermal catalytic reactor (e.g., Cu/ZnO/Al₂O₃). The reactor receives hydrogen gas generated by hydrogenation reactor 280 or methanol reactor 380 via, e.g., electrolysis. Operating conditions can include 50-250° C. and 5-50 bar (electrochemical embodiments may operate at the lower end), with an applied potential (if electrochemical) of ˜1.5-3.0 V sufficient to drive CO₂ reduction. The reactor output includes a methanol stream 286 of FIG. 2 or methanol stream 386 of FIG. 3, which can be condensed and recovered, and a light-gas overhead that can be treated (e.g., PSA or hydrogen tower) to recycle H₂ and manage CO₂/CH₄.

In certain embodiments, routine 400 further includes transferring the NH₄Cl filtrate from block 416 to an ammonia recovery column 250 and adding Ca(OH)₂ 260 and steam to ammonia recovery column 250 or ammonia recovery column 350 and removing NH₃ from the column overhead. In column 250, NH₄Cl+Ca(OH)₂ reacts to liberate NH₃ (overhead) and produce a CaCl₂) 258 bottoms stream. Column 250 can be a packed or tray stripper operated at ˜90-110° C. and ˜1-2 bar, with steam providing both sensible heat and stripping duty. Overhead NH₃ is condensed or otherwise collected as an ammonia feed stream.

Routine 400 further includes adding the NH₃ recovered from the ammonia recovery column 250 to an NH₃ absorber column 244; adding at least one of the brine solution 246, the ammonia 248, and the weak base (e.g., a primary amine) to the NH₃ absorber column; and transferring a bottom product from the NH₃ absorber column 244 to the carbonating tower 230, wherein the bottom product includes ammonia and at least one of the brine and weak base. The absorber 244 can be operated at ˜30-50° C. and ˜1-2 bar with trays or structured packing to promote gas-liquid contact; temperature control (jackets/intercoolers) may be used because NH₃ absorption is exothermic. The ammoniated/aminated brine produced at the bottom of absorber 244 provides the contacting liquid for block 412, thereby closing the NH₃ (and optional amine) recycle loop and maintaining steady-state operation of tower 230.

The present technology includes systems and methods embodied in the instructions addressed in the aspects of the present technology presented below:
Aspect 1: A method comprising receiving a flue gas at a carbon capture system, wherein the flue gas includes carbon dioxide, nitrogen, oxygen, nitrates and sulfates; introducing the flue gas into a first column of the carbon capture system, wherein the first column removes the nitrates and the sulfates from the flue gas creating a purified flue gas; removing, from the first column, the purified flue gas via an overhead stream; removing a bottoms product from the first column that includes the sulfates and nitrates from the flue gas; transferring the purified flue gas to a carbonating tower, wherein the carbonating tower has a top exit stream and a bottom exit stream; contacting, in the carbonating tower, the purified flue gas with at least one of a brine solution, ammonia, and a weak base to create at least a lean brine solution; transferring the lean brine solution from the bottom exit stream of the carbonating tower to a filter; and filtering the lean brine solution to recover NaHCO₃, wherein the primary source of carbon dioxide for the NaHCO₃ is the purified flue gas.
Aspect 2: The method of Aspect 1, further comprising transferring the NaHCO₃ to a reactor and calcining the NaHCO₃ to form substantially pure Na₂CO₃ and carbon dioxide, wherein the primary source of carbon dioxide for the Na₂CO₃ is the purified flue gas.
Aspect 3: The method of any one of Aspects 1-2, further comprising transferring the carbon dioxide to a methanol reactor and hydrogenating the carbon dioxide to produce methanol.
Aspect 4: The method of any one of Aspects 1-3, wherein the methanol is produced through electrochemical methods.
Aspect 5: The method of any one of Aspects 1-4, further comprising transferring the methanol to an aldehyde reactor and hydrogenating the methanol to produce formaldehyde.
Aspect 6: The method of any one of Aspects 1-5, wherein filtering the lean brine solution also recovers NH₄Cl.
Aspect 7: The method of any one of Aspects 1-6, further comprising transferring the NH₄Cl to an ammonia recovery column, adding Ca(OH)₂ and steam to the ammonia recovery column, and removing NH₃ from the ammonia recovery column.
Aspect 8: The method of any one of Aspects 1-7, further comprising adding the NH₃ from the ammonia recovery column to an NH₃ absorber column, adding at least one of the brine solution, the ammonia, and the weak base to the NH₃ absorber column, and transferring a bottom product from the NH₃ absorber column to the carbonating tower, wherein the bottom product includes ammonia and at least one of the brine and weak base.
Aspect 9: A carbon capture system comprising a first scrubbing column, wherein the first scrubbing column removes nitrates and sulfates from an input, wherein an overhead product from the first scrubbing column includes a flue gas after separation and a bottoms product from the first scrubbing column includes the sulfates and nitrates from the flue gas; a carbonating tower, wherein the carbonating tower removes carbon dioxide from the flue gas using at least ammonia, wherein a second overhead product from the carbonating tower includes oxygen, nitrogen and carbon monoxide and a second bottom from the carbonating tower includes a first lean caustic stream; and a separation system, wherein the separation system separates NaHCO₃ from the second bottom, wherein the primary source of carbon dioxide for the NaHCO₃ is the purified flue gas.
Aspect 10: The system of Aspect 9, further comprising a reactor, wherein the reactor calcines the NaHCO₃ to produce substantially pure Na₂CO₃ and carbon dioxide, wherein the primary source of carbon dioxide for the Na₂CO₃ is the purified flue gas.
Aspect 11: The system of any one of Aspects 9-10, further comprising a methanol reactor, wherein the methanol reactor hydrogenates the carbon dioxide to form methanol.
Aspect 12: The system of any one of Aspects 9-11, wherein the methanol reactor utilizes electrochemical reactions.
Aspect 13: The system of any one of Aspects 9-12, further comprising an aldehyde reactor, wherein the aldehyde reactor hydrogenates the methanol to produce formaldehyde.
Aspect 14: The system of any one of Aspects 9-13, wherein the separation system also separates NH₄Cl from the second bottom.
Aspect 15: The system of any one of Aspects 9-14, further comprising an ammonia recovery column, wherein the NH₄Cl, Ca(OH)₂ and steam are reacted in the ammonia recovery column to produce NH₃.
Aspect 16: The system of any one of Aspects 9-15, further comprising an NH₃ absorber column, wherein the NH₃ is reacted with at least one of the brine solution, the ammonia, and the weak base, and the carbonating tower receives a bottom product from the NH₃ absorber column, wherein the bottom product includes ammonia and at least one of the brine and weak base.
Aspect 17: A carbon capture system comprising a first scrubbing column, wherein the first scrubbing column removes nitrates and sulfates from an input, wherein an overhead product from the first scrubbing column includes a flue gas after separation and a bottoms product from the first scrubbing column includes the sulfates and nitrates from the flue gas; a carbonating tower, wherein the carbonating tower removes carbon dioxide from the flue gas using at least ammonia, wherein a second overhead product from the carbonating tower includes oxygen, nitrogen and carbon monoxide and a second bottom from the carbonating tower includes a first lean caustic stream; and a separation system, wherein the separation system separates Na₂CO₃ from the second bottom, wherein the primary source of carbon dioxide for the Na₂CO₃ is the purified flue gas.
Aspect 18: The system of Aspect 9-17, further comprising a methanol reactor, wherein the methanol reactor hydrogenates the carbon dioxide to form methanol.
Aspect 19: The system of any one of Aspects 9-18, further comprising a reactor, wherein the reactor receives the Na₂CO₃, the carbon dioxide and water to create NaHCO₄.
Aspect 20: The system of any one of Aspects 9-18, further comprising a separation system, wherein the separation system purifies the NaHCO₄.

Any of Aspects 1-20 may be combined with one another in any operable manner, unless such combination is technically inconsistent.

What is claimed is:

1. A method comprising:
receiving a flue gas at a carbon capture system, wherein the flue gas includes carbon dioxide, nitrogen, oxygen, nitrates and sulfates;
introducing the flue gas into a first column of the carbon capture system, wherein the first column removes the nitrates and the sulfates from the flue gas creating a purified flue gas,
removing, from the first column, the purified flue gas via an overhead stream;
removing a bottoms product from the first column that includes the sulfates and nitrates from the flue gas;
transferring the purified flue gas to a carbonating tower, wherein the carbonating tower has a top exit stream and a bottom exit stream;
contacting, in the carbonating tower, the purified flue gas with at least one of a brine solution, ammonia, and a weak base to create at least a lean brine solution;
transfer the lean brine solution from the bottom exit stream of the carbonating tower to a filter; and
filtering, the lean brine solution to recover NaHCO₃, wherein a primary source of carbon dioxide for the NaHCO₃ is the purified flue gas.

2. The method of claim 1, further comprising:
transferring the NaHCO₃ to a reactor; and
calcining the NaHCO₃ to form substantially pure Na₂CO₃ and carbon dioxide, wherein the primary source of carbon dioxide for the substantially pure Na₂CO₃ is the purified flue gas.

3. The method of claim 2, further comprising:
transferring the carbon dioxide to a methanol reactor; and
hydrogenating the carbon dioxide to produce methanol.

4. The method of claim 3, wherein the methanol is produced through electrochemical methods.

5. The method of claim 3, further comprising:
transferring the methanol to an aldehyde reactor; and
hydrogenating the methanol to produce formaldehyde.

6. The method of claim 1, wherein filtering the lean brine solution also recovers NH₄Cl.

7. The method of claim 6, further comprising:
transferring the NH₄Cl to an ammonia recovery column;
adding Ca(OH)₂ and steam to the ammonia recovery column; and
removing NH₃ from the ammonia recovery column.

8. The method of claim 7, further comprising:
adding the NH3 from the ammonia recovery column to a NH₃ absorber column;
adding at least one of the brine solution, the ammonia, and the weak base to the NH₃ absorber column; and
transferring a bottom product from the NH3 absorber column to the carbonating tower, wherein the bottom product includes ammonia and at least one of the brine solution and the weak base.

9. A carbon capture system comprising:
a first scrubbing column, wherein the first scrubbing column removes nitrates and sulfates from an input, wherein an overhead product from the first scrubbing column includes a flue gas after separation and a bottoms product from the first scrubbing column includes the sulfates and nitrates from the flue gas;
a carbonating tower, wherein the carbonating tower removes carbon dioxide from the flue gas using at least ammonia, wherein a second overhead product from the carbonating tower includes oxygen, nitrogen and carbon monoxide and a second bottom from the carbonating tower includes a first lean caustic stream; and
a separation system, wherein the separation system separates NaHCO₃ from the second bottom, wherein a primary source of carbon dioxide for the NaHCO₃ is the flue gas.

10. The carbon capture system of claim 9, further comprising:
a reactor, wherein the reactor calcines the NaHCO₃ to produce substantially pure Na₂CO₃ and carbon dioxide, wherein the primary source of carbon dioxide for the substantially pure Na₂CO₃ is the flue gas.

11. The carbon capture system of claim 10, further comprising:
a methanol reactor, wherein the methanol reactor hydrogenates the carbon dioxide to form methanol.

12. The carbon capture system of claim 11, wherein the methanol reactor utilizes electrochemical reactions.

13. The carbon capture system of claim 11, further comprising:
an aldehyde reactor, wherein the aldehyde reactor hydrogenates the methanol to produce formaldehyde.

14. The carbon capture system of claim 9, wherein the separation system also separates NH₄Cl from the second bottom.

15. The carbon capture system of claim 14, further comprising:
an ammonia recovery column, wherein the NH4Cl, Ca(OH)₂ and steam are reacted in the ammonia recovery column to produce NH₃.

16. The carbon capture system of claim 14, further comprising:
an NH₃ absorber column, wherein the NH₃ is reacted with at least one of a brine solution, the ammonia, a weak acid, and a weak base; and
the carbonating tower receives a bottom product from the NH₃ absorber column, wherein the bottom product includes ammonia and at least one of the brine solution and the weak base.

17. A carbon capture system comprising:
a first scrubbing column, wherein the first scrubbing column removes nitrates and sulfates from an input, wherein an overhead product from the first scrubbing column includes a flue gas after separation and a bottoms product from the first scrubbing column includes the sulfates and nitrates from the flue gas;
a carbonating tower, wherein the carbonating tower removes carbon dioxide from the flue gas using at least ammonia, wherein a second overhead product from the carbonating tower includes oxygen, nitrogen and carbon monoxide and a second bottom from the carbonating tower includes a first lean caustic stream; and
a separation system, wherein the separation system separates Na₂CO₃ from the second bottom, wherein a primary source of carbon dioxide for the Na₂CO₃ is the flue gas.

18. The carbon capture system of claim 17, further comprising:
a methanol reactor, wherein the methanol reactor hydrogenates the carbon dioxide to form methanol.

19. The carbon capture system of claim 17, further comprising:
a reactor, wherein the reactor receives the Na₂CO₃, the carbon dioxide and water to create NaHCO₄.

20. The carbon capture system of claim 19, further comprising:
a separation system, wherein the separation system purifies the NaHCO₄.
